(19) United States
(12) Patent Application Publication
(10) Pub. No.: US 2013/0211212 A1
(43) Pub. Date: Aug. 15, 2013

(54) DETERMINING THE BLOOD SUGAR LEVEL IN A PATIENT BY USING AN IMPLANTABLE SENSOR AND AN ELECTRICAL FUNCTIONAL ADHESIVE BANDAGE

(71) Applicant: Robert Bosch GmbH, (US)

(72) Inventor: Michael Stumber, Korntal-Muenchingen (DE)

(73) Assignee: Robert Bosch GmbH, Stuttgart (DE)

(21) Appl. No.: 13/760,176

(22) Filed: Feb. 6, 2013

(30) Foreign Application Priority Data
Feb. 9, 2012 (DE) 10 2012 201 892.1

(51) Int. Cl. A61B 5/145; A61B 5/1455

(52) U.S. Cl. CPC A61B 5/14532; A61B 5/1455
USPC 600/316; 600/310

(57) ABSTRACT

An implantable sensor includes a hydrogel, a glucose-binding protein and a reference molecule. The binding affinity of the reference molecule for glucose differs by at least a factor of ten from the binding affinity for glucose of the glucose-binding protein. At least one of the electromagnetic behavior and the fluorescent behavior of the glucose-binding protein and the reference molecule change when glucose is bound. An electrical functional adhesive bandage includes a measurement element for measuring at least one of electromagnetic properties and fluorescent properties. The bandage also includes a first communication element for wireless communication. Together, the implantable sensor, the bandage, and an evaluation device, which includes a computation unit, a display and a second communication element for wireless communication, form a kit for determining the blood sugar level in a patient.

This application claims priority under 35 U.S.C. §119 to patent application no. DE 10 2012 201 892.1, filed on Feb. 9, 2012 in Germany, the disclosure of which is incorporated herein by reference in its entirety.

### BACKGROUND

The present disclosure relates to an implantable sensor, an electrical functional adhesive bandage and a kit made of the implantable sensor, the electrical functional adhesive bandage and an evaluation device. The present disclosure furthermore relates to a method for determining the blood sugar level in a patient using the components of the kit according to the disclosure. Moreover, the present disclosure relates to a computer program, which executes all steps of the method according to the disclosure when it runs on a computation unit. Finally, the present disclosure relates to a computer program product with program code, which is stored on a machine-readable medium, for carrying out the method according to the disclosure when the program is executed on a computation unit.

Diabetics require regular measurement of the blood sugar level. Here, the blood sugar level is understood to mean the size of the glucose proportion in the blood. In the case of conventional methods for measuring the blood sugar, every measurement requires a blood sample to be taken. Hence a number of attempts have been made to improve or simplify the measurement of the blood sugar level in diabetics. In this case, it is desirable to find a method of measuring the blood sugar level at short time intervals which could be carried out without taking blood samples. By way of example, there are attempts in this respect to develop an implantable glucose sensor which measures the blood sugar level at regular intervals and transmits it to an evaluation unit. The latter will regularly supply the patient with current information in respect of his blood sugar level. The information could, on the one hand, be transmitted to an insulin pump for continuous regulation of the blood sugar level. On the other hand, wireless transmission of the blood sugar level to a medical monitoring system would be feasible. As a result of this medical aid could quickly be summoned in the case of critical blood sugar values. However, such implants are currently still large and very complicated.

### SUMMARY

The implantable sensor according to the disclosure comprises a hydrogel, a glucose-binding protein and a reference molecule, the glucose-affinity of which differs by at least a factor of 10 from the glucose-affinity of the glucose-binding protein. The glucose-binding protein and the reference molecule change the electromagnetic behavior and/or the fluorescent behavior thereof when glucose is bound. As a result of embedding the glucose-binding protein and the reference molecule into the hydrogel, the glucose-binding protein and the reference molecule are fixed in such a way that they cannot harm a patient into whose body the sensor is implanted, i.e. that they are biocompatible and protected from the immune system of the body, i.e. they are biostable. The hydrogel is preferably selected from the group consisting of alginate hydrogels, polyglycerylsilicate hydrogels (PSG) and zwitterionic hydrogels, in particular synthetic hydrogels of zwitterionic origin, such as e.g. sulfo betaines or carboxy betaines, or copolymers of zwitterionic monomers with hydroxyethyl methacrylate. A suitable zwitterionic hydrogel can for example be based on N-(3-sulfopropyl)-N-(methacryloyloxyethyl)-N,N-dimethylammoniobetaine (SMADB). These hydrogels are very well suited to immobilizing biological material and have good biocompatible properties. By way of example, a change in the fluorescent behavior of the glucose-binding protein and the reference molecule when glucose is bound can be brought about by binding on one or more fluorescent chemical groups. Preferably two fluorescent groups are bound on in order to enable a Forster resonant energy transfer (FRET) when there is a change in conformation of the glucose-binding protein. By way of example, a change in the electromagnetic behavior of the glucose-binding protein and the reference molecule when glucose is bound can be brought about by binding on one or more metallic nano-beads or nanoparticles at a first position of the protein or the molecule and at least one electrically conductive or strongly polarizable ligand at a second position of the protein or molecule. The nanoparticle is preferably a magnetic nanoparticle. Furthermore, for steric reasons, it is preferable for the diameter of the nanoparticle not to exceed 100 nm.

So that the reference molecule allows reliable referencing, it is preferable for the glucose-binding protein and the reference molecule to be bonded by means of the same binding mechanism to one or more fluorescent groups or one or more nano-beads, nanoparticles and electrically conductive or strongly polarizable ligands. It is furthermore preferable for the denaturation behavior (sensitivity of the natural secondary or tertiary structure of the protein with respect to environmental effects such as heat, acid, salts) of the glucose-binding protein and of the reference molecule to be substantially the same. In order to allow a distinction to be made between glucose-binding protein and reference molecule by an electromagnetic measurement or a fluorescence measurement, it is preferable, according to the disclosure, for these to be bound to different nano-beads, or nanoparticles and electrically conductive or strongly polarizable ligands or for the fluorescence maxima thereof to lie at different wavelengths. Furthermore, in order to enable a simple distinction between the two substances, it is preferable for the glucose- binding protein and the reference molecule to be arranged in different regions of the hydrogel. In order to examine the fluorescent behavior more easily, it is furthermore preferable for the implantable sensor to comprise a reflective, preferably biocompatible, layer, which amplifies a fluorescence signal by reflection.

The electrical functional adhesive bandage comprises a measurement element for measuring electromagnetic properties and/or fluorescent properties, and a first communication element for wireless communication. In order to measure fluorescent properties, the measurement element can, for example, be a fluorescence-exciting LED or laser diode, which is connected to a photodiode which can capture the light signal from an excited fluorescence. In order to measure electromagnetic properties, a measurement element can be a device for emitting a radiofrequency pulse and for examining frequency-dependencies of an electromagnetic response. Furthermore, use can be made of a device for measuring a permittivity or a tunneling current. The first communication element preferably enables encrypted radio communication by means of Bluetooth, ZigBee or a proprietary standard.

In addition to the implantable sensor and the electrical functional adhesive bandage, the kit according to the disclosure comprises an evaluation device, which comprises a computation unit, a display and a second communication element for wireless communication. The second communication element is preferably configured in such a way that it can communicate wirelessly with the first communication element by means of a common standard. By way of example, the evaluation device can be a smartphone or a reader.

In the method according to the disclosure for determining the blood sugar level in a patient, a sensor is initially implanted under the skin of the patient, said sensor comprising a hydrogel and a glucose-binding protein, the latter changing the electromagnetic behavior or the fluorescent behavior thereof when glucose is bound. An electrical functional adhesive bandage according to the disclosure is subsequently positioned above the sensor on the skin of the patient. The electromagnetic properties and/or the fluorescent properties of the glucose-binding protein are measured by means of the measurement element. Since the hydrogel renders it possible for a chemical equilibrium to be set between glucose in the blood of the patient and glucose which is bound to the glucose-binding protein and the electromagnetic signal or the fluorescence signal allows conclusions to be drawn as to how much glucose is bound to the glucose-binding protein, the measurement signal allows conclusions to be drawn in respect of the blood sugar level in the patient. The measurement result is transmitted to a second communication element of an evaluation device by means of the first communication element in the electrical functional adhesive bandage. The blood sugar level in the patient is now calculated in a computation unit of the evaluation device from the measurement result and a reference value established for this patient. Here, the reference value can be used for compensating for the fading or aging of a fluorescent dye which is bound to the glucose-binding protein, for monitoring the protein state or aging processes of the glucose-binding protein, for compensating for a drift and for calibration purposes. If the implanted sensor is an implanted sensor according to the disclosure, it is possible to determine the reference value by virtue of the electromagnetic properties and/or the fluorescent properties of the reference molecule being measured by means of the measurement element. Thus, there is internal referencing. This is advantageous, in particular, for compensating for an aging of the fluorescent dye or for compensating for a drift. Alternatively, the blood sugar level in the patient can be determined by examining a blood sample and a blood sugar level established thus can be used as reference value for a number of implementations of the method. In particular, examining one blood sample on a monthly, quarterly or semi-annual basis suffices for this purpose. This means a significantly lower burden on the patient due to blood samples than in the case of the conventional blood sugar determination, which requires several blood samples to be taken daily.

The computer program according to the disclosure enables the implementation of the method according to the disclosure in a conventional evaluation device, which comprises a computation unit, such that, for example, a conventional smartphone can be used in a method according to the disclosure by uploading the computer program according to the disclosure. The computer program product according to the disclosure, with program code, serves to this end, which computer program code is stored on a machine-readable medium, for carrying out the method when the product is executed on a computation unit.

### BRIEF DESCRIPTION OF THE DRAWINGS

Exemplary embodiments of the disclosure are illustrated in the drawings and explained in more detail in the following description.

FIG. 1 shows the arrangement of an implantable sensor of an electrical functional adhesive bandage and an evaluation device when carrying out a method as per one embodiment of the disclosure.

FIG. 2 shows the change in the conformation of a glucose-binding protein when binding a glucose molecule and the change in the fluorescence emission spectrum thereof resulting therefrom.

FIG. 3 shows the FRET response to a physiological change in the glucose concentration of the glucose-binding protein.

### DETAILED DESCRIPTION

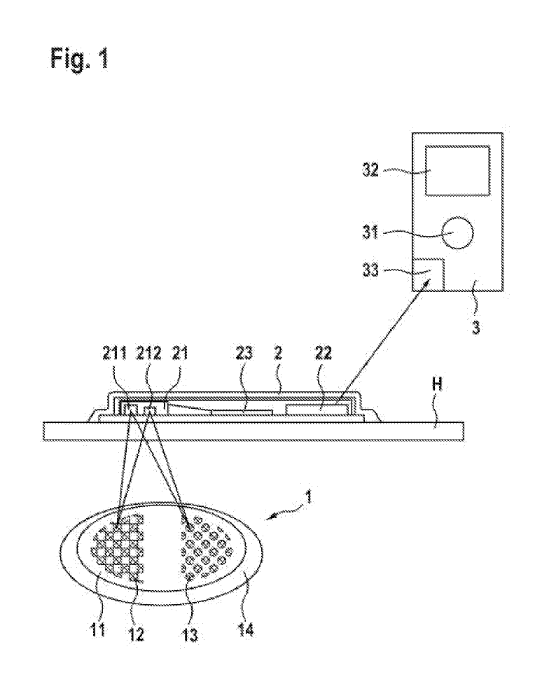

FIG. 1 shows an implantable sensor 1 as per one embodiment of the disclosure, an electrical functional adhesive bandage 2 as per one embodiment of the disclosure, an evaluation device 3 and the arrangement thereof when carrying out a method as per one embodiment of the disclosure.

The implantable sensor comprises a main body made of a hydrogel 11. By way of example, the hydrogel 11 can be an alginate hydrogel, a hydrogel which can be obtained by virtue of a calcium (II) salt, e.g. calcium chloride, calcium carbonate or Na₂CaEDTA, being added to a solution of alginic acid. A glucose-binding protein 12 and a reference protein 13 are arranged in two different regions of the hydrogel 11. By way of example, the glucose-binding protein 12 is the protein GBPfluo 5.4, which has been provided with two fluorescent groups and can be obtained by expressions from myoblasts C2C12 (rat) G3. By way of example, the reference protein 13, which is arranged in another region of the hydrogel 11, is a protein which has likewise been provided with two fluorescent groups, the binding-affinity for glucose (“glucose-affinity”) of which protein differs by at least a factor of 10 from the glucose-affinity of GBPfluo 5.4 and the denaturation behavior of which substantially corresponds to that of GBPfluo 5.4. The implantable sensor 1 is implanted subcutaneously, i.e. under the skin H of a patient. On the side thereof facing away from the skin H, the sensor has a biocompatible reflective layer 14.

A soft, flat and flexible functional adhesive bandage 2 is stuck onto the skin H of the patient above the implantable sensor 1. Said functional adhesive bandage comprises a measurement element 21 for measuring fluorescent properties of the glucose-binding protein 12 and of the reference molecule 13. The measurement element 21 consists of a laser diode 211 and a photodiode 212 with an optical filter. The electrical functional adhesive bandage 2 furthermore has a first wireless radio communication element 22, which is connected to the measurement element 21 by means of electronics 23. The patient has a smartphone as evaluation device 3. The latter has a microchip as computation unit 31, a display 32 and a second wireless radio communication element 33 for wireless communication with the first wireless communication element 22.

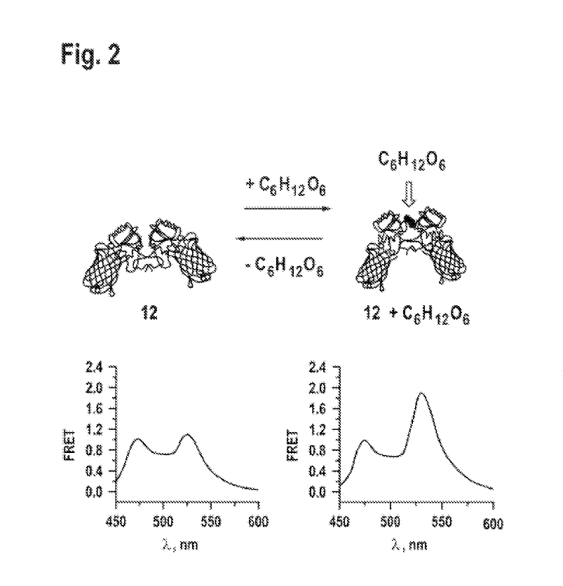

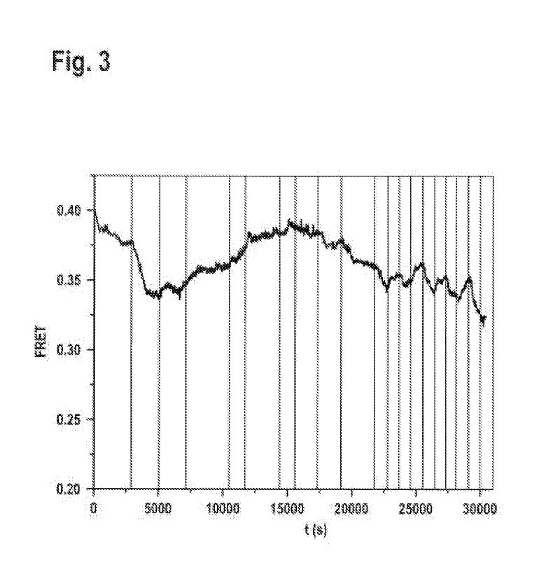

In order to determine the blood sugar level in a patient suffering from diabetes, the sensor 1 is initially implanted under the skin H of the patient. The electrical functional adhesive bandage 2 is subsequently stuck onto the skin H of the patient above the sensor 1. The functional adhesive bandage 2 can be replaced if necessary. The electronics 23 activate the laser diode 211 at regular intervals, for example a number of times per hour. Said laser diode transmits a laser beam to the implanted sensor 1 through the skin H of the patient and successively excites the glucose-binding protein 12 and the reference molecule 13 to fluoresce. A FRET fluorescence signal is subsequently detected by the photodiode 212. FIG. 2 shows the conformation change of the glucose-binding protein 12 when binding glucose (C₆H₁₂O₆) and the change in the FRET signal resulting therefrom. FIG. 3, in an exemplary fashion, shows the intensity profile of the FRET signal at the wavelength of maximum fluorescence intensity over a time interval of 500 hours. The electronics 23 transmit the measurement result from the measurement element 21 to the communication element 22, which transmits said measurement result wirelessly to the second communication element 33 of the evaluation device 3. There, the measurement result is transmitted to the computation unit 31, which calculates the blood sugar level in the patient from the fluorescence signal of the glucose-binding protein 12 and the fluorescence signal of the reference molecule 13 as reference, compensating for an aging of the fluorescent dye and compensating for the drift, and outputs said blood sugar level via the display 32.

In another embodiment of the disclosure, an electric nanoparticle with a diameter of less than 100 μm and an electrically conductive ligand are attached to the glucose-binding protein instead of the two fluorescent groups, which nanoparticle and ligand exhibit a modified response behavior to an electric radiofrequency pulse. In this embodiment of the disclosure, rather than the laser diode 211 and the photodiode 212, the measurement element 21 has a device which can emit an electromagnetic radiofrequency pulse and can, very sensitively, detect and filter or process the electromagnetic response from the glucose-binding protein and from the reference molecule to the radiofrequency pulse.

What is claimed is:

1. An implantable sensor comprising:
a hydrogel;
a glucose-binding protein; and
a reference molecule,
wherein a binding affinity for glucose of the reference molecule differs by at least a factor of ten from a binding affinity for glucose of the glucose-binding protein,
wherein at least one of the electromagnetic properties and the fluorescent properties of the glucose-binding protein changes when the glucose-binding protein is bound to glucose, and
wherein at least one of the electromagnet properties and the fluorescent properties of the reference molecule changes when the reference molecule is bound to glucose.

2. The implantable sensor according to claim 1, wherein the glucose-binding protein and the reference molecule are arranged in different regions of the hydrogel.

3. The implantable sensor according to claim 1, further comprising a reflective layer.

4. An electrical functional adhesive bandage comprising:
a measurement element configured to measure at least one of electromagnetic properties and fluorescent properties; and
a first communication element configured to communicate wirelessly.

5. A kit comprising:
an implantable sensor including a hydrogel, a glucose-binding protein, and a reference molecule, wherein a binding affinity for glucose of the reference molecule differs by at least a factor of ten from a binding affinity for glucose of the glucose-binding protein, wherein at least one of the electromagnetic properties and the fluorescent properties of the glucose- binding protein changes when the glucose-binding protein is bound to glucose, and wherein at least one of the electromagnet properties and the fluorescent properties of the reference molecule changes when the reference molecule is bound to glucose;
an electrical functional adhesive bandage including a measurement element configured to measure at least one of electromagnetic properties and fluorescent properties, and a first communication element configured to communicate wirelessly; and
an evaluation device including a computation unit, a display, and a second communication element configured to communicate wirelessly.

6. A method for determining the blood sugar level in a patient comprising:
implanting a sensor under skin of the patient, wherein said sensor includes a hydrogel and a glucose-binding protein, wherein at least one of electromagnetic properties and fluorescent properties of the glucose- binding protein changes when glucose is bound;
positioning an electrical functional adhesive bandage on the skin of the patient, wherein the adhesive bandage includes a measurement element configured to measure at least one of electromagnetic properties and fluorescent properties, wherein the adhesive bandage includes a first communication element configured to communicate wirelessly;
measuring at least one of the electromagnetic properties and the fluorescent properties of the glucose-binding protein with the measurement element;
wirelessly transmitting a measurement result to a second communication element of an evaluation device via the first communication element; and
calculating the blood sugar level in the patient from the measurement result and a reference value established for the patient.

7. The method according to claim 6, further comprising determining the reference value by considering at least one of the electromagnetic properties and the fluorescent properties of the reference molecule measured by the measurement element.

8. The method according to claim 6, further comprising determining the blood sugar level in the patient by examining a blood sample to establish a blood sugar level and using the blood sugar level as the reference value for a number of implementations of the method.

9. The method according to claim 6, further comprising running a computer program on a computation unit to implement the method.

10. The method according to claim 6, further comprising running a computer program of a computer program product, the computer program stored on a machine-readable medium, on a computation unit to implement the method.
